(19) United States
(12) Patent Application Publication
(10) Pub. No.: US 2023/0206585 A1
(43) Pub. Date: Jun. 29, 2023

(54) DRUG LIST DISPLAY METHOD AND DRUG LIST

(71) Applicant: FUJIFILM Toyama Chemical Co., Ltd., Tokyo (JP)

(72) Inventors: Hirona YUMBE, Tokyo (JP); Shigetoshi ISHIKAWA, Tokyo (JP)

(73) Assignee: FUJIFILM Toyama Chemical Co., Ltd., Tokyo (JP)

(21) Appl. No.: 18/173,065

(22) Filed: Feb. 23, 2023

Related U.S. Application Data
(63) Continuation of application No. PCT/JP2021/030956, filed on Aug. 24, 2021.

(30) Foreign Application Priority Data
Aug. 31, 2020 (JP) 2020-145764

(51) Int. Cl. G06V 10/20; G06T 7/00; G06F 3/0482

(52) U.S. Cl. CPC G06V 10/255; G06T 7/0012; G06F 3/0482

(57) ABSTRACT

A drug list display method includes: obtaining a first image of drugs captured from one side of the drugs; clipping from the first image, areas where the respective drugs are displayed to obtain first clipped images; displaying the first clipped images in a list form in a certain order in a first area on a display in such a manner that one or more of the first clipped images may be designated; determining correct drugs for first clipped images designated by a user; and displaying information on the correct drugs determined for the respective first clipped images in a second area on the display different from the first area, wherein the first clipped images and the corresponding information on the correct drugs are displayed in association with each other.

### CROSS-REFERENCE TO RELATED APPLICATIONS

The present application is a Continuation of PCT International Application No. PCT/JP2021/030956 filed on Aug. 24, 2021 claiming priority under 35 U.S.C § 119(a) to Japanese Patent Application No. 2020-145764 filed on Aug. 31, 2020. Each of the above applications is hereby expressly incorporated by reference, in its entirety, into the present application.

### BACKGROUND OF THE INVENTION

### 1. Field of the Invention

The present invention relates to a drug list display method and a drug list, and particularly to a technique which compares a drug to be identified with a drug serving as a candidate.

### 2. Description of the Related Art

There has been known an apparatus which identifies tablets from images obtained by imaging the tablets, analyzes images of tables to be distinguished among the identified tablets to extract tablets serving as candidates, and displays the extracted candidate tablets in a list.

Japanese Patent Application Laid-Open No. 2020-011122 (hereinafter referred to as “Patent Literature 1”), Japanese Patent Application Laid-Open No. 2019-076787 (hereinafter referred to as “Patent Literature 2”) and W02017/183533 (hereinafter referred to as “Patent Literature 3”) each describe an apparatus which, when drugs having not been distinguished yet are selected from among drugs displayed in a first part of a tablet image distinguishing window, displays the selected drugs in a second part in an enlarged manner, and displays candidate drugs for the selected drugs in a third part on a list.

Japanese Patent Application Laid-Open No. 2017-158743 (hereinafter referred to as “Patent Literature 4”) describes an apparatus which, in a case where abnormality is present in a captured image when identification information on a drug is recognized based on the captured image of the drug, displays an identification mark for identifying a part where the abnormality is present in the captured image so as to overlap the captured image.

Japanese Patent Application Laid-Open No. 2013-137775 (hereinafter referred to as “Patent Literature 5”) describes an apparatus which, when required items are input and a search button is operated in a process of distinguishing drugs brought by a patient, searches a database about drugs, and displays a list of drugs in a search result in a drug search result field.

### CITATION LIST

Patent Literature 1: Japanese Patent Application Laid-Open No. 2020-011122

Patent Literature 2: Japanese Patent Application Laid-Open No. 2019-076787

Patent Literature 3: WO2017/183533

Patent Literature 4: Japanese Patent Application Laid-Open No. 2017-158743

Patent Literature 5: Japanese Patent Application Laid-Open No. 2013-137775

### SUMMARY OF THE INVENTION

In some cases, a tablet has information such as an imprint (impression) and a printed mark that enables to identify the tablet only on one surface of the tablet, and does not have information on the other surface. There has been a problem that, when a plurality of tablets including such a tablet are identified, identifiable drugs and unidentifiable drugs coexist so that it is difficult to understand which drugs are identified.

The present invention has been made in view of such situations, and aims to provide a drug list display method that display imaged drugs and determined drugs in a contrasted manner (contrastably), and the drug list.

In order to achieve the object described above, an aspect of a drug list display method includes: a first image obtaining step of obtaining a first image of drugs captured from a side of one surface of the drugs; a first clipping step of clipping from the first image, areas where the respective drugs are displayed to obtain first clipped images; a clipped image displaying step of displaying the first clipped images in a list form in a certain order in a first area on a display in such a manner that one or more of the first clipped images may be designated; a determining step of determining correct drugs for first clipped images designated by a user; and a drug information displaying step of displaying information on the correct drugs determined for the respective first clipped images in a second area on the display different from the first area, wherein the first clipped images and the corresponding information on the correct drugs are displayed in association with each other.

According to this aspect, the first clipped images are displayed in a list form in the first area, and information on the correct drugs are displayed in a list form in the second area in association with the respectively corresponding first clipped images. Consequently, the imaged drugs and the determined drugs may be displayed in a contrasted manner.

Preferably, in the certain order, first clipped images which represent information contributing to determination of correct drugs are superior (higher ranked) among the first clipped images, and first clipped images which represent no information contributing to determination of correct drugs are inferior (lower ranked). Accordingly, the list may be displayed in such a manner that drugs facilitating determination are higher ranked, and drugs not facilitating determination are lower ranked.

Preferably, among the first clipped images which represent the information contributing to determination of correct drugs, a first clipped image having information contributing to the determination with a relatively higher degree, becomes superior in the certain order. Accordingly, the list may be displayed in such a manner that the drugs more facilitating determination are higher ranked.

Preferably, the drug list display method further includes a candidate information displaying step of displaying information on candidate drugs for determining the correct drugs for the one or more designated first clipped images, wherein the determining step determines candidate drugs selected by the user from among the candidate drugs, as the correct drugs for the one or more designated first clipped images. Accordingly, the correct drugs may be determined among the candidate drugs.

Preferably, the candidate information displaying step displays the information on the candidate drugs in a third area between the first area and the second area on the display. Accordingly, the information on the candidate drugs can be displayed in a list form at a position allowing the user to easily view the information on the candidate drugs.

Preferably, the information on the candidate drugs includes at least one of: names of the drugs; master images of the drugs; and images of PTP (Press Through Package) sheets of the drugs. Accordingly, the correct drugs may be appropriately determined.

Preferably, the drug list display method further includes: a second image obtaining step of obtaining a second image of the drugs captured from a side of another surface opposite to the one surface; and a second clipping step of clipping areas of the respective drugs from the second image to obtain a second clipped images, wherein the clipped image displaying step displays, instead of the first clipped images, the second clipped images in a list form in a certain order in such a manner that one or more of the displayed images may be designated. Accordingly, drugs imaged from the opposite side and determined drugs may be displayed in a contrasted manner.

Preferably, the drug list display method further includes an association step of selecting from among the second clipped images, one or more second clipped images for which it can be confirmed from the one or more second clipped images themselves, that drugs imaged in the one or more second clipped images are the correct drugs determined in the first clipped images, and associating the one or more second clipped images with the first clipped images, wherein the clipped image displaying step displays the second clipped images corresponding to the first clipped images, and information on the correct drugs corresponding to the first clipped images, in association with each other. Accordingly, the association between the imaged drugs and the determined drugs may be recognized by the user.

Preferably, the association step confirms drugs whose correct drugs are determined in the first clipped images, based on at least one of: printed marks or imprints on the another side of the drugs; colors of the drugs; and shapes of the drugs. Accordingly, it is possible to appropriately confirm drugs whose correct drugs are determined in the first clipped images.

Preferably, the determining step displays: the first clipped images or the second clipped images; and associated information which is associated with the first clipped images or the second clipped images and the information on the correct drugs. Accordingly, the association between the first clipped images or the second clipped images and the correct drugs may be recognized by the user.

Preferably, the associated information includes marks indicating a determined order. Accordingly, the determined order, number of same drugs and the like may be recognized by the user.

Preferably, the clipped image displaying step displays the printed marks and the imprints in the first clipped images and the second clipped images, in an emphasized manner. Accordingly, the plurality of drugs may be displayed in a list form in such a manner that the user can recognize the drugs easily.

Preferably, in a case where the first clipped images or the second clipped images displayed in a list form in the first area are scrolled, the drug information displaying step scrolls the information on the correct drugs displayed in a list form in the second area in synchronization with the scrolling. Accordingly, the clipped images and the information on the correct drugs may be always displayed in a list form in an associated manner.

This aspect also includes a program causing a computer to execute the drug list display method described above. This aspect may also include a non-transitory computer-readable storage medium on which the program is recorded. According to this aspect, the first clipped images may be are displayed in a list form in the first area, and information on correct drugs may be displayed in a list form in the second area in association with the respective first clipped images. Consequently, the imaged drugs and the determined drugs may be displayed in a contrasted manner.

In order to achieve the object described above, an aspect of a drug list is a drug list to be displayed on a display, wherein: first clipped images are displayed in a list form in a first area in a certain order, the first clipped images obtained by clipping areas where drugs are respectively displayed from a first image of the drugs captured from one side of the drugs; information on correct drugs is displayed in a second area different from the first area, the correct drugs respectively determined for the first clipped images; and the first clipped images and information on correct drugs respectively corresponding to the first clipped images, are displayed in association with each other.

According to this aspect, the first clipped images are displayed in a list form in the first area, and information on the plurality of correct drugs are displayed in a list form in the second area in association with the respective first clipped images. Consequently, the imaged drugs and the determined drugs may be displayed in a contrasted manner.

According to the present invention, imaged drugs and determined drugs may be displayed in a contrasted manner.

### BRIEF DESCRIPTION OF THE DRAWINGS

FIG. 1 is a block diagram showing an example of a hardware configuration of a drug identifying system.

FIG. 2 is a plan view showing three drug packages.

FIG. 3 is a plan view showing a schematic configuration of an imaging apparatus.

FIG. 4 is a side view showing the schematic configuration of the imaging apparatus.

FIG. 5 is a flowchart showing steps of a drug identifying method.

FIG. 6 shows an example of a screen displayed on a display.

FIG. 7 shows an example of a screen displayed on the display.

FIG. 8 shows an example of a screen displayed on the display.

FIG. 9 shows an example of a screen displayed on the display.

FIG. 10 shows an example of a screen displayed on the display.

FIG. 11 shows an example of a screen displayed on the display.

FIG. 12 shows an example of a screen displayed on the display.

FIG. 13 shows an example of a screen displayed on the display.

FIG. 14 shows an example of a screen displayed on the display.

FIG. 15 shows an example of a screen displayed on the display.

FIG. 16 shows an example of a screen displayed on the display.

FIG. 17 shows an example of a screen displayed on the display.

FIG. 18 shows an example of a screen displayed on the display.

FIG. 19 shows an example of a screen displayed on the display.

FIG. 20 shows an example of a screen displayed on the display.

FIG. 21 shows an example of a screen displayed on the display.

FIG. 22 shows an example of a screen displayed on the display.

### DESCRIPTION OF THE PREFERRED EMBODIMENTS

Hereinafter, referring to the accompanying drawings, preferred embodiments of the present invention are described in detail.

### Configuration of Drug Identifying System

A drug identifying system 10 is a system that captures an image of drugs, which are imaging objects (object to be imaged), and determines correct drugs in the drugs in the captured image. The drug identifying system 10 may be a system that distinguishes drugs, or a system that checks (audit) drugs. Distinguishing of the drugs means specifying types of the drugs based on appearances of the drugs. Checking (audit) of the drugs means confirming whether the drugs are dispensed in conformity with a prescription. Here, identification is a concept including distinguishing and checking.

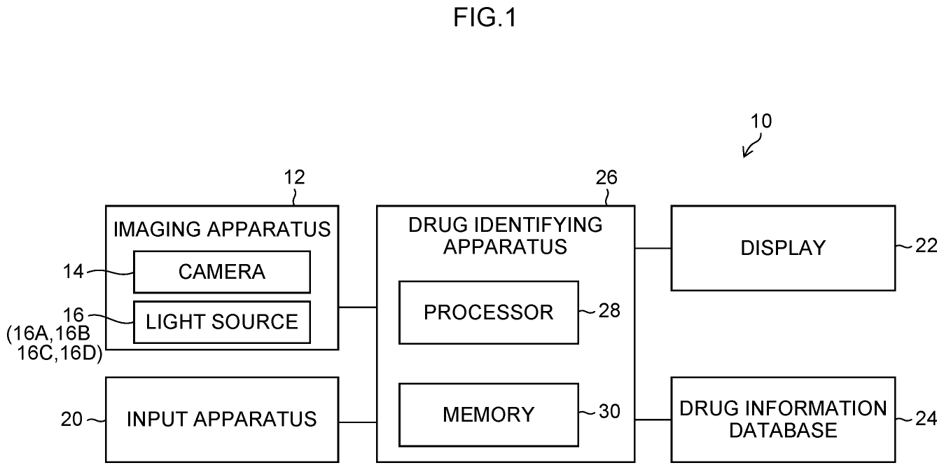

FIG. 1 is a block diagram showing an example of the hardware configuration of the drug identifying system 10. As shown in FIG. 1, the drug identifying system 10 includes an imaging apparatus 12, an input apparatus 20, a display 22, a drug information database 24, and a drug identifying apparatus 26.

The imaging apparatus 12 includes a camera 14, and a light source 16. The camera 14 is a so-called digital camera that includes an object lens, not shown, and an image pickup element, not shown. The camera 14 captures a color image of an imaging object. The image pickup element may be a CMOS (complementary metal oxide semiconductor) type or a CCD (charge coupled device) type. The camera 14 includes a data processing circuit that generates digital image data from an analog signal on a pixel-by-pixel basis read from the image pickup element. The camera 14 may capture a still image, or capture a moving image at a certain frame rate.

The light source 16 includes four light sources 16A, 16B, 16C and 16D. The light sources 16A, 16B, 16C and 16D are LED (Light Emitting Diode) light sources. The light sources 16A, 16B, 16C and 16D emit illumination light of visible light toward the imaging object respectively in directions different from each other.

The input apparatus 20 is an input interface for allowing a user to input various types of information and a desired instruction into the drug identifying apparatus 26. The input apparatus 20 includes a pointing device such as a mouse, and an input device such as a keyboard.

The display 22 is a display apparatus for allowing the user to view information such as image data. The display 22 functions as a part that displays a screen required for operations on the input apparatus 20 and achieves a GUI (Graphical User Interface). The display 22 displays a drug recognition result and the like. A touch panel display that integrally includes an input apparatus 20 and a display 22 may be applied to the drug identifying system 10.

The drug information database 24 is a database including a mass storage apparatus. The drug information database 24 manages drug information that is detailed information on each drug. The drug information on each drag includes: a GS1 code (identification code according to GS1 standards); a name of the drug, master images of the drug, and images of PTP (Press Through Package) sheets of the drag.

The drug identifying apparatus 26 is made up of a general-purpose computer, for example. The drug identifying apparatus 26 obtains image data from the imaging apparatus 12. The drug identifying apparatus 26 obtains an instruction and the like input by the user, via the input apparatus 20. The drug identifying apparatus 26 displays information such as image data, on the display 22. Furthermore, the drug identifying apparatus 26 obtains desired drug information from the drug information database 24.

The drug identifying apparatus 26 includes a processor 28 and a memory 30. The processor 28 executes instructions stored in the memory 30.

The hardware structure of the processor 28 is that of any of various processors as described below. The various processors include: a CPU (Central Processing Unit) that is a general-purpose processor that executes software (program) and serves as various types of functional units; a GPU (Graphics Processing Unit) dedicated to image processing; a PLD (Programmable Logic Device) such as FPGA (Field Programmable Gate Array), which is a processor whose circuit configuration can be changed after production; and a dedicated electric circuit that is a processor having a circuit configuration designed solely for executing specific processes, such as an ASIC (Application Specific Integrated Circuit).

One processing unit may be made up of one of these various types of processors, or two or more processors of the same type or different types (e.g., a plurality of FPGAs, or a combination of a CPU and an FPGA, or a combination of a CPU and a GPU). A plurality of functional units may be made up of a single processor. Examples where the plurality of functional units are made up of a single processor are described below. In the first example, a combination of one or more CPUs and software constitutes one processor, and the processor functions as the plurality of functional units, as typified by a computer such as a client or a server. In the second example, a processor is used to achieve the functions of the entire system including a plurality of functional units by a single IC (Integrated Circuit) chip, as typified by SoC (System On Chip) or the like. As described above, each type of functional units has a hardware structure made up of one or more of the various types of processors.

More specifically, the hardware structures of the various types of processors are electric circuits (circuitry) achieved by combining circuit elements, such as semiconductor elements.

The memory 30 stores instructions to be executed by the processor 28. The memory 30 includes a RAM (Random Access Memory) and a ROM (Read Only Memory), not shown. The processor 28 uses the RAM as a work area and executes software, using various types of programs including a drug identifying program, and parameters, which are stored in the ROM, and executes various types of processes of the drug identifying apparatus 26 using parameters stored in the ROM and the like.

### Configuration of Imaging Apparatus

Imaging objects to be imaged by the imaging apparatus 12 are, for example, a plurality of drugs per does. The plurality of drugs may be in a drug package (chartula), or not in a drug package (i.e., loose pills).

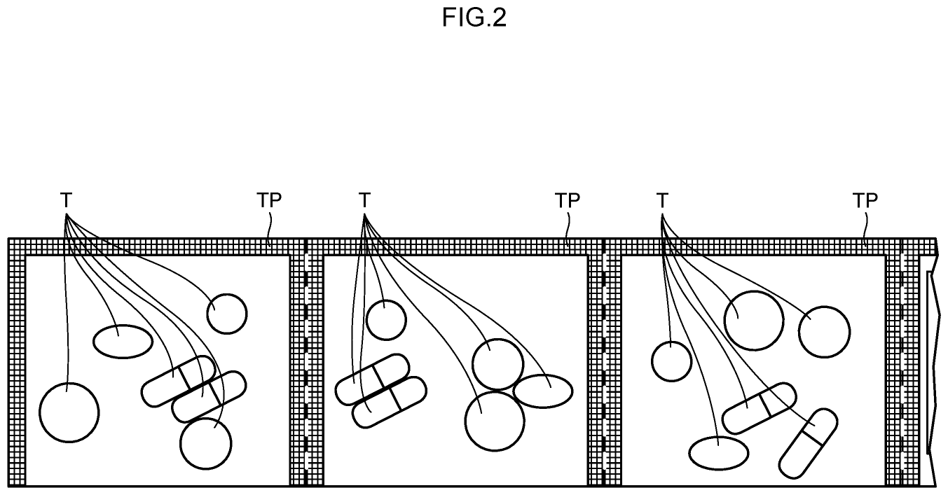

FIG. 2 is a plan view showing three drug packages in each of which a plurality of drugs are collectively packed. In each drug package TP shown in FIG. 2, six drugs T are packed, for example. The drug packages TP are joined to form a strip shape, and perforated lines are formed to allow each drug package TP to be separated. Here, the imaging apparatus 12 images the drugs T for each drug package TP.

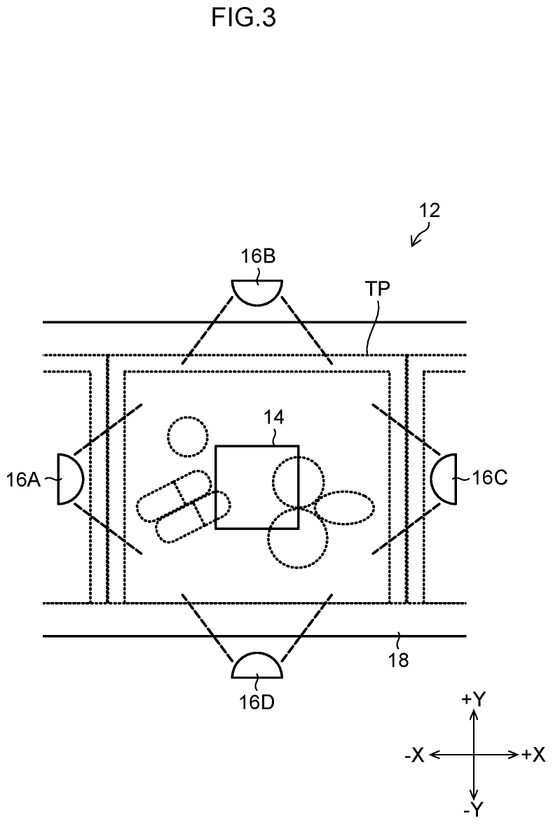

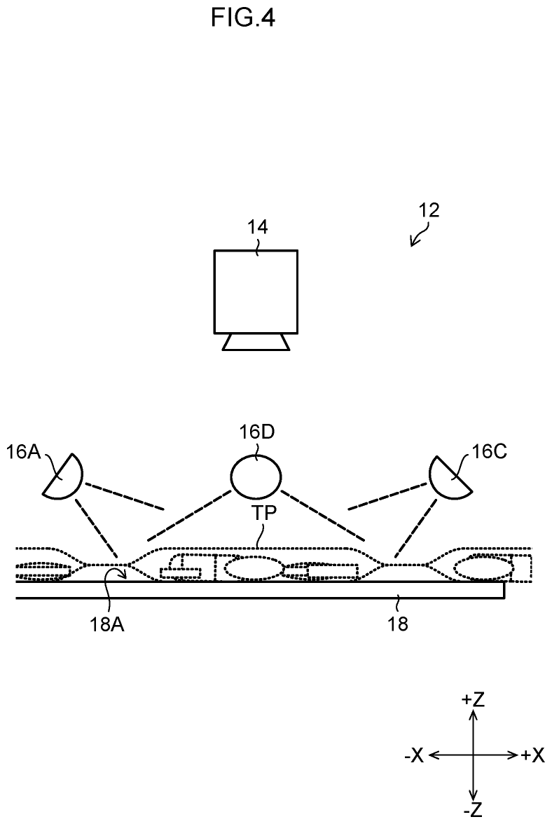

FIG. 3 is a plan view showing a schematic configuration of the imaging apparatus 12. FIG. 4 is a side view showing the schematic configuration of the imaging apparatus 12. The imaging apparatus 12 includes a stage 18 on which the drug packages TP are mounted. The stage 18 is supported by a support member, not shown, with a flat mounting surface 18A which is in parallel with a horizontal plane (XY plane). The drug packages TP are mounted on the mounting surface 18A of the stage 18.

The camera 14 is supported by a support member, not shown, at a position apart in a +Z direction by a certain distance from the center of the mounting surface 18A of the stage 18, with the optical axis being directed toward the mounting surface 18A.

The light sources 16A, 16B, 16C and 16D are respectively supported by support members, not shown, at positions apart from the stage 18 in the +Z direction by certain distances. The light source 16A is arranged at a position apart from the center of the stage 18 in the −X direction by a certain distance. The light source 16B is arranged at a position apart from the center of the stage 18 in the +Y direction by a certain distance. The light source 16C is arranged at a position apart from the center of the stage 18 in the +X direction by a certain distance. The light source 16D is arranged at a position apart from the center of the stage 18 in the −Y direction by a certain distance.

The light sources 16A, 16B, 16C and 16D respectively emit illumination lights toward the stage 18 from directions inclined to the +Z direction. Light emission of the light sources 16A, 16B, 16C and 16D is individually controlled by the drug identifying apparatus 26.

Imaging is performed as follows. First, the imaging apparatus 12 causes the camera 14 to image a first surface of the drug package TP. The imaging apparatus 12 sequentially causes the light sources 16A, 16B, 16C and 16D to emit lights so that the camera 14 captures an image in a state where only the light source 16A is emitting light, an image in a state where only the light source 16B is emitting light, an image in a state where only the light source 16C is emitting light, and an image in a state where only the light source 16D is emitting light. Subsequently, the imaging apparatus 12 causes the camera 14 to capture an image in a state where the light sources 16A, 16B, 16C and 16D are simultaneously emitting lights.

Next, the imaging apparatus 12 causes the camera 14 to image a second surface that is a surface opposite to the first surface of the drug package TP. In order to image the second surface, a user turns the drug package TP upside down, and mounts the drug package TP on the mounting surface 18A of the stage 18 again. Turning upside down may be performed by an automatic inverting mechanism, not shown. The imaging by the camera 14 is similar to the case for the first surface.

In the four images captured while the light sources 16A, 16B, 16C and 16D sequentially emitting lights, the respective illumination directions are different from each other. Accordingly, in a case where an imprint is formed on a surface of a drug T, shades are formed differently from each other in the four images, due to the imprint (convexo-concave). These four captured images are used to generate an imprint image where the imprint on the surface of the drug is emphasized. The four captured images may be used to generate an image where the printed mark on the surface of the drug is emphasized.

Specifically, the imprint is a groove that is a depressed area on the surface of the drug T, and indicates identification information on the drug T. The groove is not limited to what is formed by engraving the surface, and may be what is formed by pressing the surface. Alternatively, the imprint may include a score or the like accompanied by no identifying function.

A single image captured while the light sources 16A, 16B, 16C and 16D simultaneously emitting lights, is an image without luminance unevenness. Accordingly, the image is used when the image of the surface of the drug T (drug image) is clipped. The imprint image is superimposed on the image.

Note that the order of imaging one drug package TP, and the number of captured images are not limited to those in the example described above. Further, the images are captured in a state of a dark room. The light emitted to the drug package TP during imaging is only the illumination light from the light source 16. Accordingly, in the images captured as described above, the background is black, and the area of each drug T has the color of the drug T.

### Drug Identifying Method

A drug identifying method using the drug identifying system 10 is described. The drug identifying method is achieved by the processor 28 executing a drug identifying program stored in the memory 30. The drug identifying program may be provided through a non-transitory computer-readable storage medium. In this case, the drug identifying apparatus 26 may read the drug identifying program from the non-transitory storage medium, and store the program in the memory 30.

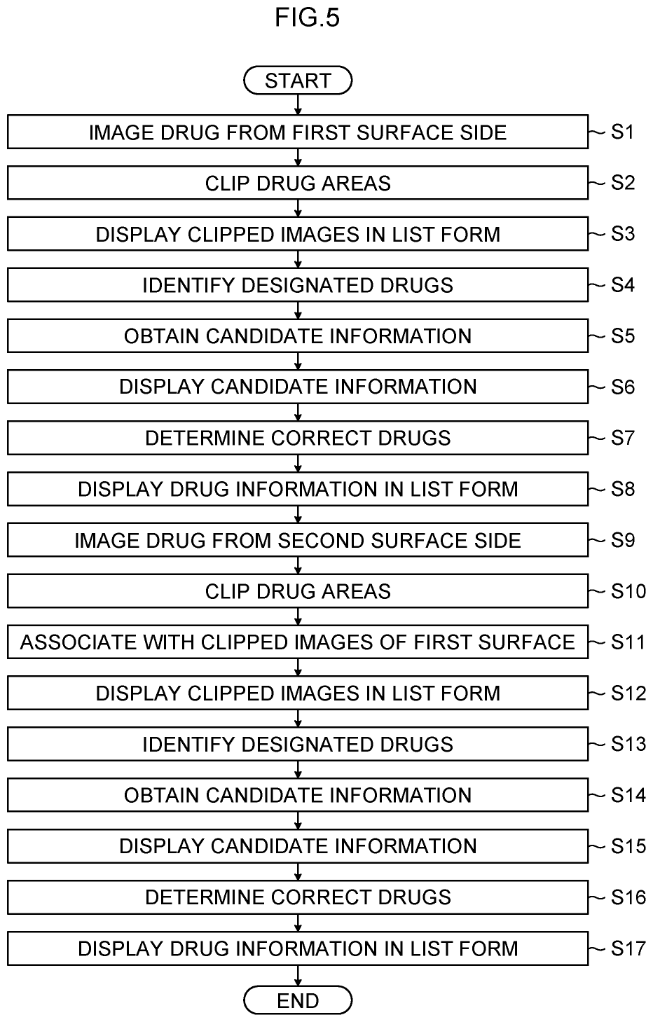

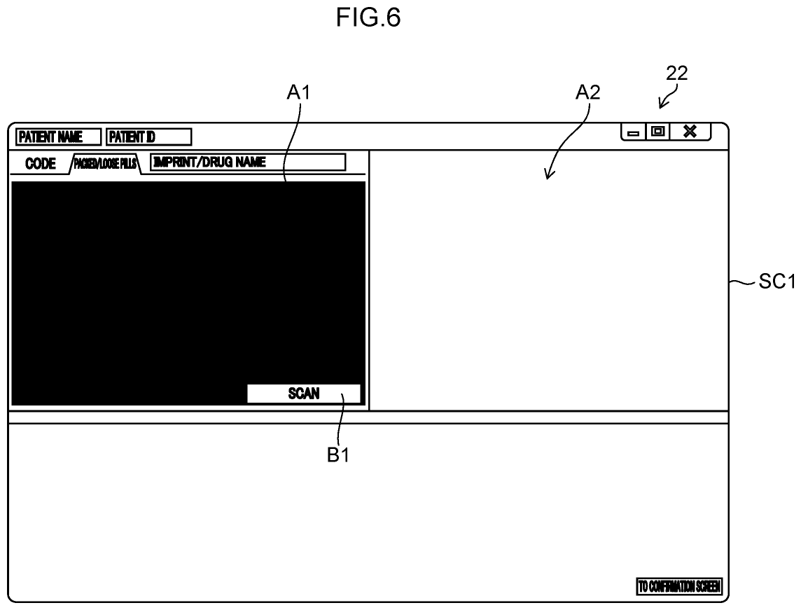

FIG. 5 is a flowchart showing steps of the drug identifying method. FIGS. 6 to 22 show examples of screens displayed on the display 22. An example of imaging a drug package TP including drugs T and identifying the drugs T, is herein described.

FIG. 6 shows a screen SC1 on the display 22 when the drug identifying method begins. As shown in FIG. 6, the drug identifying apparatus 26 divides the screen SC1 into an upper left part as a first area A1, and an upper right part as a second area A2. The drug identifying apparatus 26 arranges a button B1 at the bottom of the first area A1. The button B1 is a button for starting to image the drug package TP.

The user mounts the drug package TP to be imaged on the mounting surface 18A of the stage 18, with the first surface of the drug package TP facing in the +Z direction. Subsequently, when the user operates the button B1 using the input apparatus 20, the drug identifying apparatus 26 causes the camera 14 to image the drug package TP containing the drugs T, and obtains first images where the drug package TP is imaged from a side of the first surface (example of one surface) (step S1: example of first image obtaining step).

Here, the camera 14 captures four images in accordance with control by the drug identifying apparatus 26 while the light sources 16A, 16B, 16C and 16D sequentially emitting lights, and captures one image while the light sources 16A, 16B, 16C and 16D simultaneously emitting lights. The drug identifying apparatus 26 obtains five images in total, as the first images.

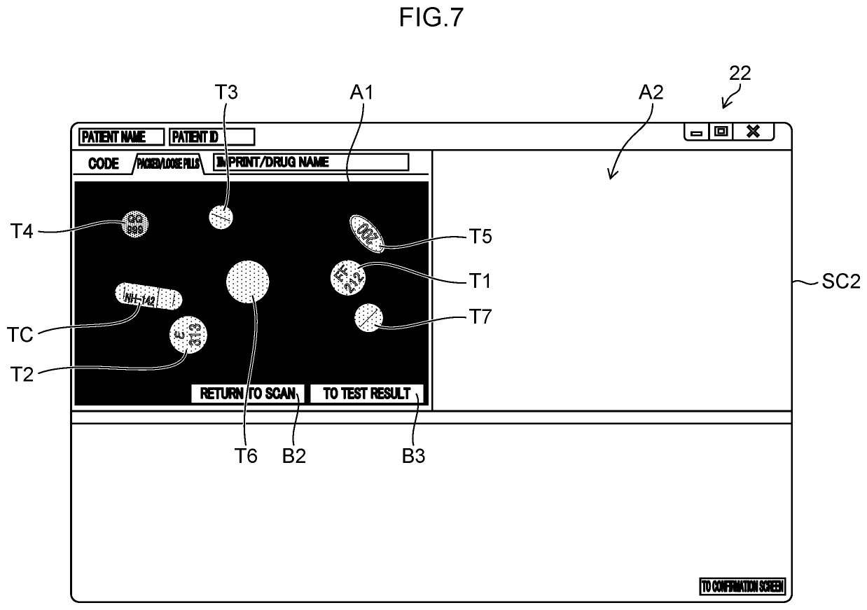

Next, the drug identifying apparatus 26 displays, on the display 22, the images taken by imaging the drugs T. Here, the drug identifying apparatus 26 displays the first image captured in a state where the light sources 16A, 16B, 16C and 16D are simultaneously emitting lights, in the first area A1 of the display 22. FIG. 7 shows a screen SC2 on the display 22 at this time point. As shown in FIG. 7, on the screen SC2, the first image where seven tablets T1 to T7 and one capsule TC are imaged as drugs T, is displayed in the first area A1.

The drug identifying apparatus 26 arranges a button B2 and a button B3, instead of the button B1, at the bottom of the first area A1. The button B2 is a button for re-executing imaging the drug package TP. When the user operates the button B2 using the input apparatus 20, the drug identifying apparatus 26 returns the processing to the process in step S1.

The button B3 is a button for identifying positions where the respective drugs are displayed, from the image displayed on the display 22. When the user operates the button B3 using the input apparatus 20, the drug identifying apparatus 26 identifies the positions where the respective drugs T are displayed, from the image displayed on the display 22. The positions where the drugs T are displayed are identified as coordinate positions on a screen on the display 22. The drug identifying apparatus 26 displays, on the display 22, rectangular frames that surround areas of the respective drugs T whose positions are identified. Furthermore, the drug identifying apparatus 26 generates imprint images of the respective drugs T from the four images which are captured while the light sources 16A, 16B, 16C and 16D sequentially emitting lights in step S1, and superimposes the generated imprint images respectively on the areas of the respective drugs T in the first image displayed in the first area. The drug identifying apparatus 26 performs a process of emphasizing the printed parts on the drugs, based on the image captured while the light sources 16A, 16B, 16C and 16D simultaneously emitting lights.

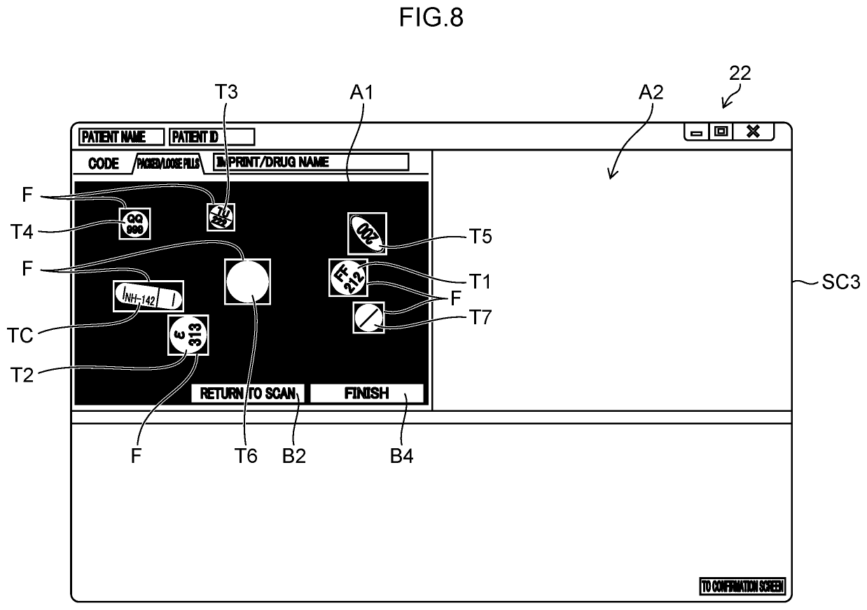

FIG. 8 shows a screen SC3 on the display 22 at this time point. As shown in FIG. 8, rectangular frames F that surround the areas of the seven tablets T1 to T7 and the capsule TC are displayed on the screen SC3. The imprints on the tablets T1 to T7 and the printed mark on the capsule TC are displayed in an emphasized manner.

At the bottom of the first area A1 on the screen SC3, a button B4 is arranged instead of the button B3. The button B4 is a button for completing determination of correct drugs for the first image. The button B4 is described later.

Next, the drug identifying apparatus 26 clips from the first image, areas where the respective drugs T are displayed, to obtain first clipped images (step S2: example of first clipping step). Furthermore, the drug identifying apparatus 26 displays the first clipped images in the first area A1 on the display in a list form in a certain order, in such a manner that the first clipped images may be respectively designated (step S3: example of clipped image displaying step).

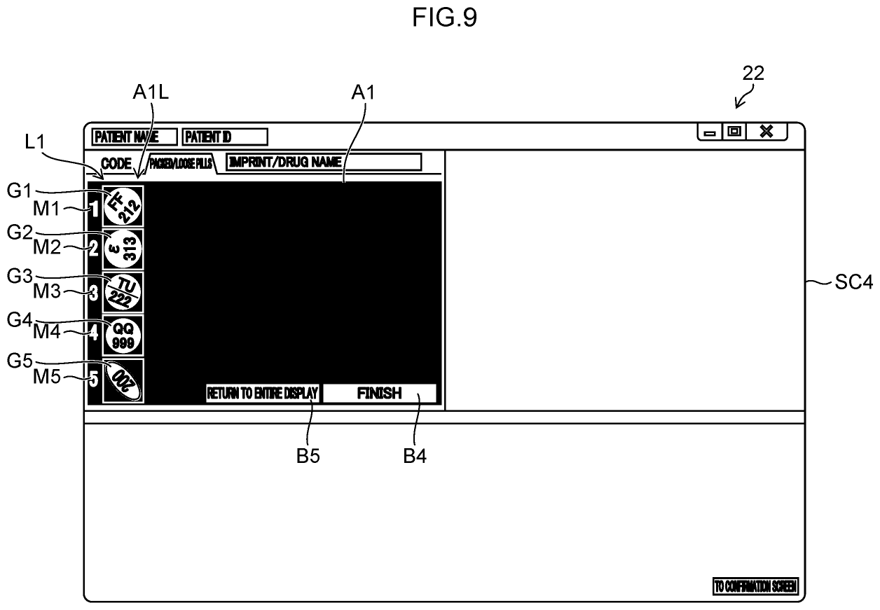

FIG. 9 shows a screen SC4 on the display 22 at a time point when the process in step S3 is finished. As shown in FIG. 9, in a left area A1L in the first area A1 on the screen SC4, the first clipped images are arranged along the vertical direction of the display 22 and displayed in a list L1. Here, the list L1 displays the first clipped images G1, G2, G3, G4 and G5 of the respective tablets T1, T2, T3, T4 and T5, but does not display the clipped images of the tablets T6 and T7 and the capsule TC. The list L1 may be scrolled as described later, so as to display the clipped images of the tablets T6 and T7 and the capsule TC.

On the screen SC4, marks indicating numbers (ranking numbers) according to the certain order, are displayed adjacent to the respective first clipped images. Specifically, the first clipped images G1, G2, G3, G4 and G5 are respectively assigned with the respective marks M1, M2, M3, M4 and M5 sequentially indicating numbers of 1, 2, 3, 4 and 5.

Preferably, as for the numbers (ranking numbers) according to the order, rankings of first clipped images that represent information contributing to determination of the correct drugs among the plurality of first clipped images are superior (higher ranked), rankings of first clipped images that represent no information contributing to determination of the correct drugs are inferior (lower ranked). Preferably, among the first clipped images that represent information contributing to determination of the correct drugs, a first clipped image that represent information contributing to determination of the correct drug with a relatively higher degree, is superior (higher ranked). In the example of the screen SC4, a first clipped image with a higher ranking has a smaller number, and is arranged at an upper position on the display 22. The degree of contributing to determination of the correct drugs can be extracted based on analysis of the first clipped images by the drug identifying apparatus 26.

Note that in a case where first clipped images of the drugs T have a high possibility that their drugs T are the same drug with each other, the same order is assigned to the first clipped images and the first clipped images are grouped and displayed (so as to be displayed in parallel).

At the bottom of the first area A1 on the screen SC4, a button B5 is arranged instead of the button B2. The button B5 is a button for displaying the entire first image in the first area A1. When the user operates the button B5 using the input apparatus 20, the drug identifying apparatus 26 returns the displayed state of the display 22 to the screen SC3.

The drug identifying apparatus 26 identifies a drug designated by the user among the drugs in the first clipped images displayed in the list on the display 22 (step S4). For example, the user designates any one first clipped image from among the first clipped images displayed in the list, using the pointing device of the input apparatus 20. The drug identifying apparatus 26 identifies a drug designated by the user, based on the first clipped image designated through the pointing device.

Subsequently, the drug identifying apparatus 26 obtains, from the drug information database 24, information on candidate drugs in order to determine a correct drug corresponding to the designated drug which is identified in step S4 (step S5). The drug identifying apparatus 26 extracts candidate drugs for the designated drug by template matching between the designated drug and the master images stored in the drug information database 24. The drug identifying apparatus 26 may extract the candidate drugs for the designated drug by drug identifying AI (Artificial Intelligence) which uses images of the designated drug as inputs. In this explanation, the drug identifying apparatus 26 extracts three candidate drugs, and obtains information on the extracted three candidate drugs from the drug information database 24.

Next, the drug identifying apparatus 26 displays, in the first area A1 on the display 22, information on the candidate drugs obtained in step S5, to be used for determining the correct drug for the designated first clipped image (step S6: example of candidate information displaying step).

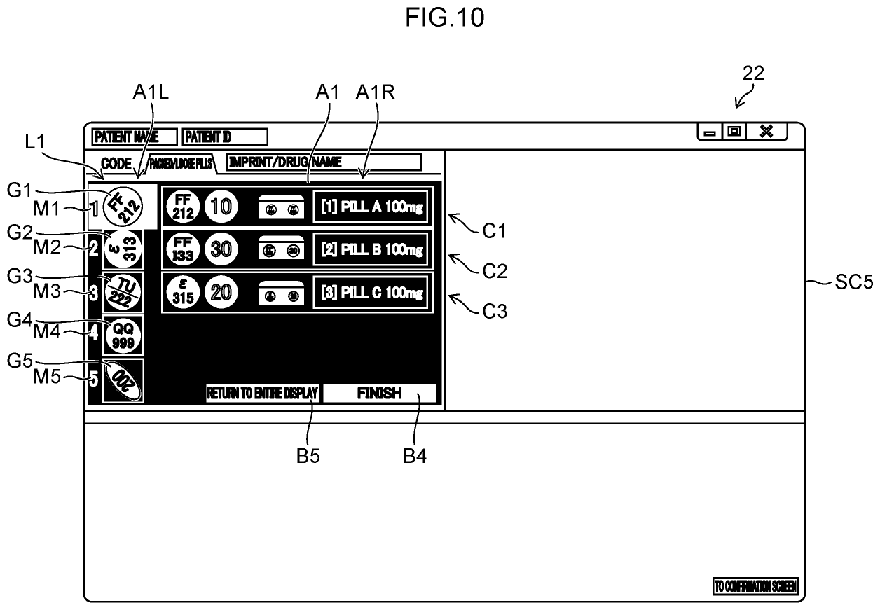

FIG. 10 shows a screen SC5 on the display 22 at a time point when the process in step S6 is finished. On the screen SC5, the first clipped image G1 of the tablet T1 is selected by the user, and the tablet T1 is identified as a designated drug. The periphery of the first clipped image G1 of the tablet T1, which is the designated drug, is displayed with a color different from the colors of the other parts in the first area A1, thus clearly indicating that the tablet T1 of the first clipped image G1 is the designated drug.

On the screen SC5, information on candidate drugs for the tablet T1, which is the designated drug, is arranged along the vertical direction of the display 22 and displayed in a right area MR (example of a third area) in the first area A1 on the display 22 so as not to overlap the list L1. In the example of the screen SC5, information on the candidate drugs C1, C2 and C3 is included as the information on the candidate drugs.

The information on each of the candidate drugs includes at least one of: a name of the drug; master images of the drug; and an image of PTP sheet of the drug. Preferably, the master images of a drug include: a front image and a back image.

On the screen SC5, the user may select information on one of the three candidate drugs C1, C2 and C3, using the pointing device of the input apparatus 20. The drug identifying apparatus 26 determines, as a correct drug corresponding to the designated drug, the candidate drug selected by the user from among the candidate drugs (step S7: determining step). Furthermore, in a case where correct drugs have been respectively determined for a plurality of first clipped images, the drug identifying apparatus 26 displays information on the correct drugs determined for the respective first clipped images in a list form in the second area A2 which is different from the first area A1, on the display (step S8: drug information displaying step). Here, the drug identifying apparatus 26 displays the first clipped images and the respectively corresponding information on correct drugs determined for the first clipped images, in association with each other. Further, the drug identifying apparatus 26 displays information which is respectively associated with both the first clipped images and the information on the correct drugs (example of determining step).

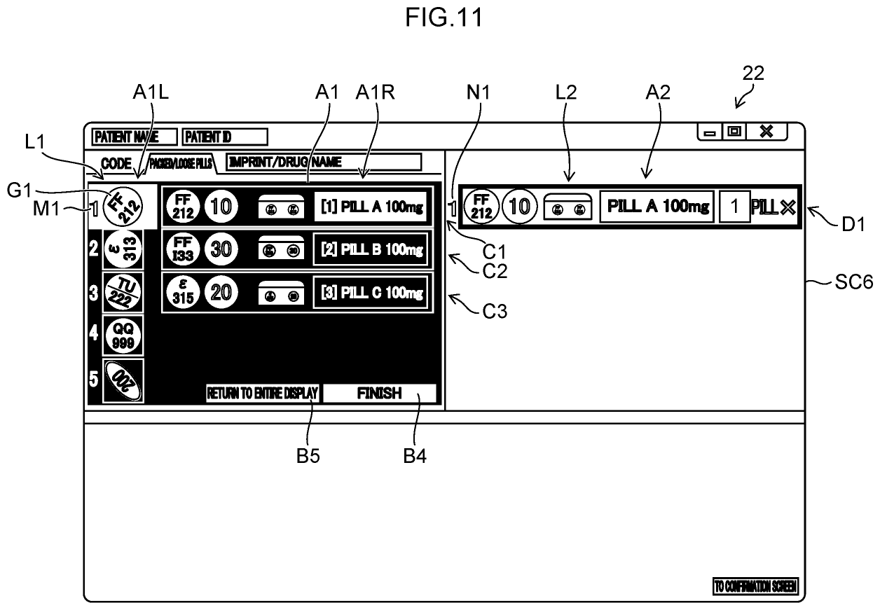

FIG. 11 shows a screen SC6 on the display 22 at this time point. As shown in FIG. 11, on the screen SC6, the color of the field of the drug name of the candidate drug C1 selected by the user is changed so as to clearly indicate that the candidate drug C1 is the selected drug.

On the screen SC6, the candidate drug C1 selected by the user is displayed as a correct drug D1 in a list L2 in the second area A2 on the display 22. The information on the correct drug D1 includes at least one of: a name of the drug; a master images of the drug; and an image of PTP sheet of the drug. Preferably, the master images of each drug include a front image and a back image. In this example, in a case where the same correct drug is determined for some of the first clipped images, the number of the first clipped images for which the same correct drug is determined is further displayed as the information on the correct drug D1. Consequently, the user may recognize the number of the same correct drugs included in the first image.

On the screen SC6, the first clipped image G1 of the tablet T1 and the information on the correct drug D1, which is the correct drug corresponding to the tablet T1, are respectively arranged in the left area A1L and the second area A2 of the first area A1 on the display 22 at the same position in the vertical direction, and are thus arranged side by side in the lateral direction (example of display in association with each other). Furthermore, on the screen SC6, a mark N1 (example of associated information) indicating the same number as that of a mark M1 assigned to the first clipped image G1 is displayed as the information on the correct drug D1. Accordingly, the first clipped image and the information on the correct drug can be displayed in a contrasted manner (contrastably). Here, displaying of the first clipped image G1 and the information on the correct drug D1 in association with each other, is not limited to displaying in such a manner that the first clipped image G1 and the information on the correct drug D1 are precisely arranged side by side. As long as association can be recognized by the user, the first clipped image G1 and the information on the correct drug D1 may be arranged in such a manner that the first clipped image G1 is deviated from the information on the correct drug D1 to some extent, or the first clipped image G1 and the information on the correct drug D1 may be arranged apart from each other.

Subsequently, when the user operates the button B4 using the input apparatus 20, the drug identifying apparatus 26 completes determination of the correct drug for the first image, and proceeds the processing to a process in step S9.

Note that when the user determines the correct drug corresponding to a drug T other than the tablet T1, the processes in steps S4 to S8 may be re-executed.

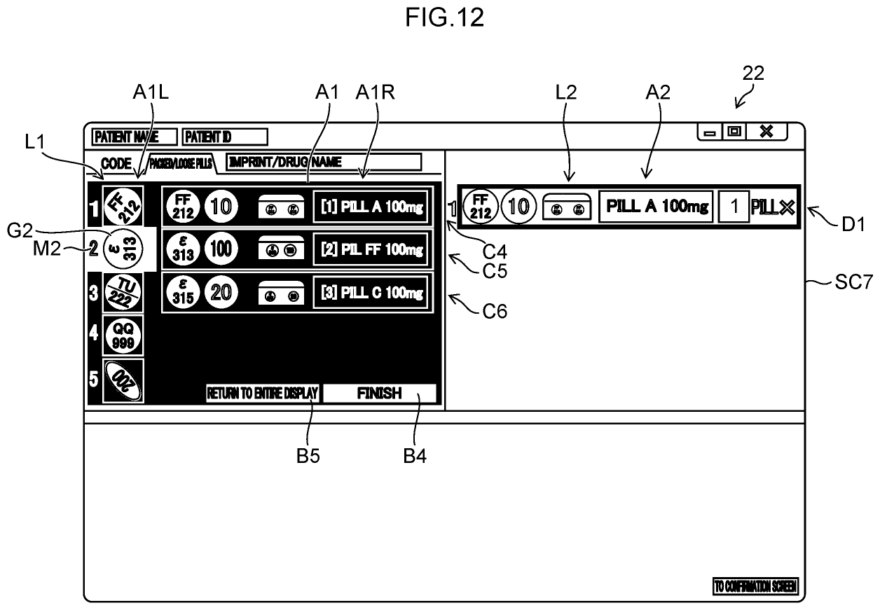

FIG. 12 shows a screen SC7 on the display 22 at a time point when the first clipped image G2 of the tablet T2 is selected by the user in a state where the screen SC6 is displayed, and the tablet T2 is identified as a second designated drug. As shown in FIG. 12, on the screen SC7, a periphery of the first clipped image G2 of the tablet T2, which is identified as the designated drug, is displayed with a color different from the colors of the other parts in the first area A1. On the screen SC7, information on candidate drugs for the tablet T2 is displayed in the right area MR in the first area A1. The information on the candidate drugs includes information on candidate drugs C4, C5 and C6.

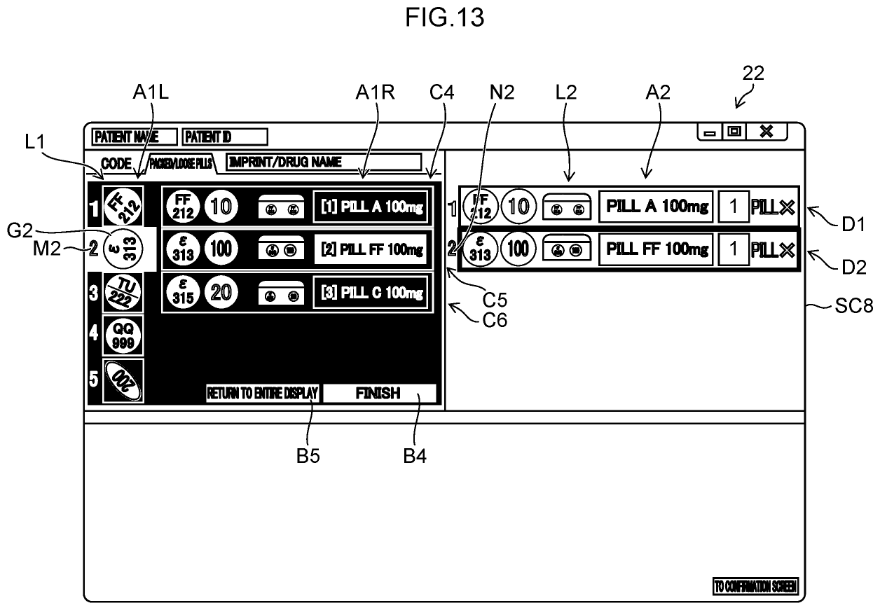

FIG. 13 shows a screen SC8 on the display 22 at a time point when the user selects the candidate drug C5 in a state where the screen SC7 is displayed. As shown in FIG. 13, a color of a field of a drug name of the candidate drug C5, which is selected by the user, is changed on the screen SC8.

On the screen SC8, the candidate drug C5 selected by the user is displayed as a correct drug D2 corresponding to the tablet T2 in the list L2 in the second area A2 on the display 22. Further, on the screen SC8, the first clipped image G2 of the tablet T2 and information on the correct drug D2, which is a correct drug corresponding to the tablet T2, are arranged side by side in the lateral direction of the display 22. Furthermore, on the screen SC8, a mark N2 indicating the same number as that of the mark M2, which is assigned to the first clipped image G2, is displayed in the information on the correct drug D2.

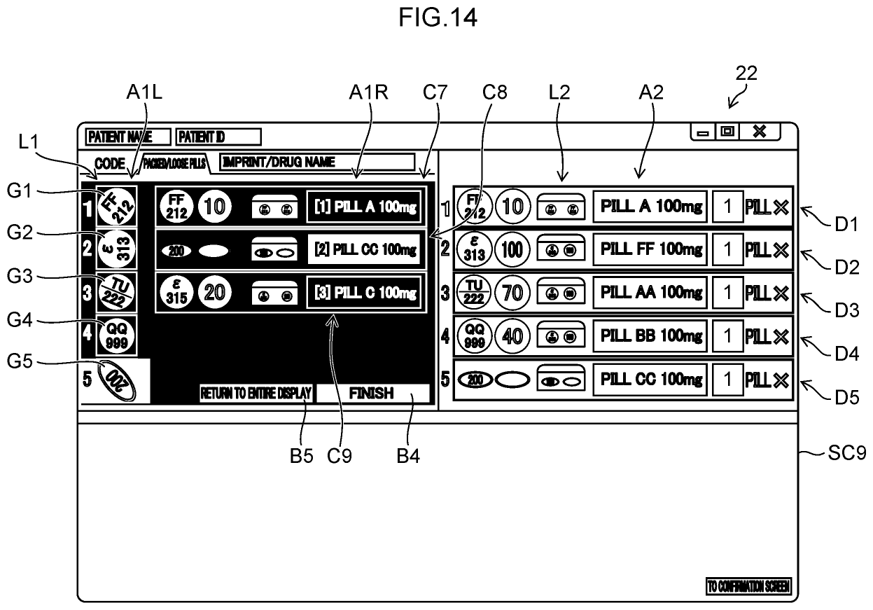

FIG. 14 shows a screen SC9 on the display 22 at a time point when the user has determined correct drugs corresponding to the tablets T3 and T4, and has identified the tablet T5 as a designated drug, and further the user selects a candidate drug C8 from among correct drugs C7, C8 and C9. On the screen SC9, the correct drugs D1 to D5 for the tablets T1 to T5 are displayed in a list form so as to be arranged along the vertical direction on the display 22. On the screen SC9, the first clipped images G1 to G5 in the list L1 and the information on the correct drugs D1 to D5 in the list L2, which respectively correspond to the first clipped images G1 to G5, are arranged side by side in the lateral direction. The list L1 and the list L2 displayed in this manner, constitute a drug list. According to the drug list, the clipped images of the drugs and the information on the determined drugs can be displayed in a contrasted manner (in a comparable manner).

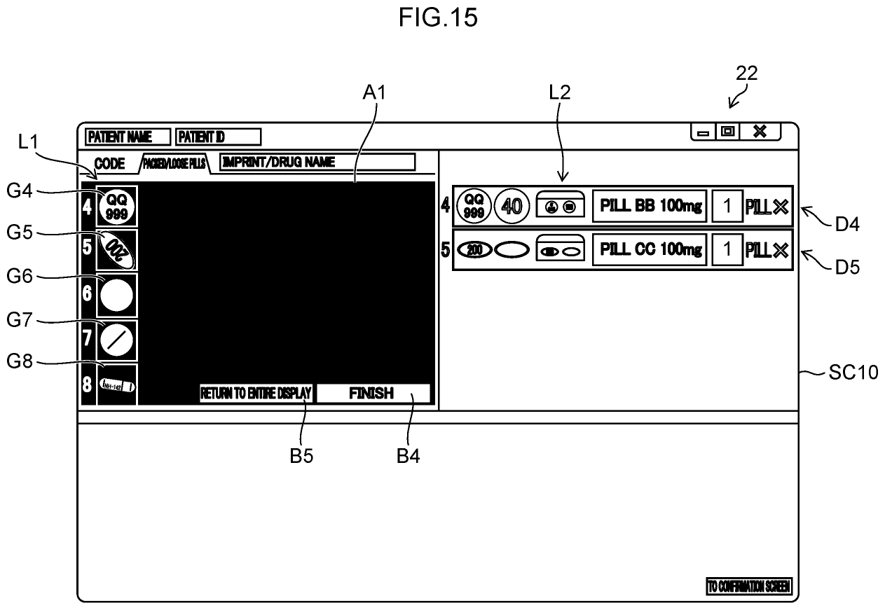

FIG. 15 shows a screen SC10 on the display 22 at a time point when the user scrolls the list L1 upward on the display 22 using the input apparatus 20 in a state where the screen SC9 is displayed. The screen SC10 is in a state where the list L1 has been scrolled so that the first clipped images G1 to G3 are hidden from the first area A1, and the first clipped images G4, G5, G6, G7 and G8 are displayed. Here, the list L2 is scrolled by the same amount in synchronization with the scrolling of the list L1. Consequently, the correct drugs D1 to D3 in the list L2 are hidden from the second area A2. Accordingly, the first clipped images G4 and G5, and the information on the correct drugs D4 and D5, which respectively correspond to the first clipped images G4 and G5, are arranged side by side in the lateral direction. Consequently, the first clipped image and the information on the correct drug can be displayed in a contrasted manner.

The example where the list L1 is scrolled is herein described. Also in a case where the list L2 is scrolled, the list L1 is scrolled by the same amount in synchronization with the scrolling of the list L2. Consequently, the first clipped images G4 and G5, and the information on the correct drugs D4 and D5, which correspond to each other, are arranged side by side in the lateral direction.

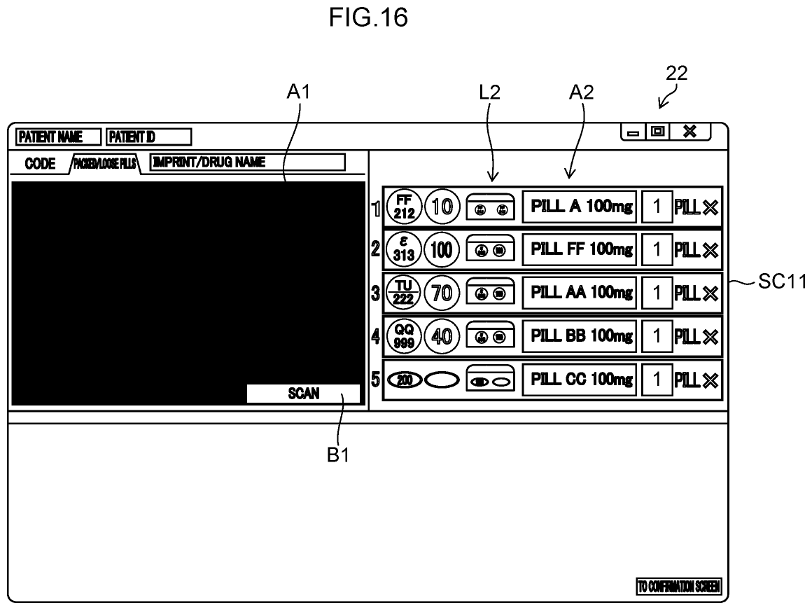

FIG. 16 shows a screen SC11 on the display 22 at a time point (time point when the button B4 is operated) when identification of the correct drugs for the first image is finished. At the bottom of the first area A1 on the screen SC11, the button B1 is arranged. The list L2 is displayed in the second area A2.

The user turns upside down the drug package TP mounted on the mounting surface 18A of the stage 18, and mounts the drug package TP on the mounting surface 18A again in such a manner that the second surface, which is opposite to the first surface, of the drug package TP faces in the +Z direction. Subsequently, when the user operates the button B1 using the input apparatus 20, the drug identifying apparatus 26 causes the camera 14 to image the drug package TP containing the drugs T to obtain second images which are images of the drug package TP imaged from a side of the second surface (example of another surface opposite to one surface) (step S9: example of second image obtaining step). Here, the camera 14 captures four images in accordance with control by the drug identifying apparatus 26 while the light sources 16A, 16B, 16C and 16D sequentially emitting lights. Further, the camera 14 captures one image in a state where the light sources 16A, 16B, 16C and 16D are simultaneously emitting lights. The drug identifying apparatus 26 obtains the total five images as the second images.

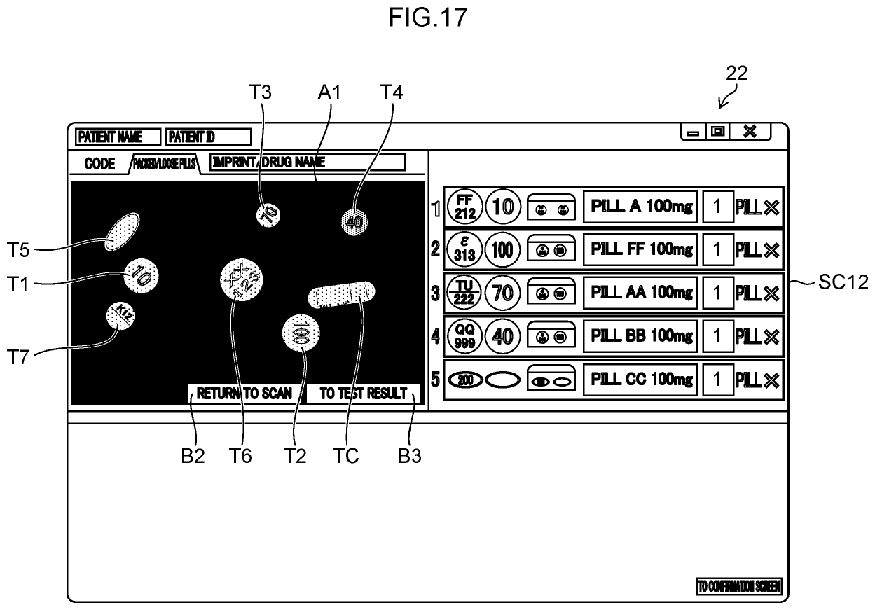

Next, the drug identifying apparatus 26 displays, on the display 22, the second images obtained by imaging the drugs T. Here, the drug identifying apparatus 26 displays the second image captured in a state where the light sources 16A, 16B, 16C and 16D are simultaneously emitting lights, in the first area A1 of the display 22. FIG. 17 shows a screen SC12 on the display 22 at this time point. As shown in FIG. 17, on the screen SC12, the second image where the seven tablets T1 to T7 and one capsule TC are imaged, is displayed in the first area A1.

The drug identifying apparatus 26 arranges a button B2 and a button B3 at the bottom of the first area A1, instead of the button B1. When the user operates the button B3 using the input apparatus 20, the drug identifying apparatus 26 identifies the positions where the respective drugs T are displayed, from the image displayed on the display 22. The drug identifying apparatus 26 displays, on the display 22, rectangular frames F that surround areas of the respective drugs T whose positions are identified. Furthermore, the drug identifying apparatus 26 generates imprint images of the respective drugs T from the four images captured while the light sources 16A, 16B, 16C and 16D sequentially emitting lights in step S9, and superimposes the generated imprint images on the areas of the respective drugs T in the second image displayed in the first area.

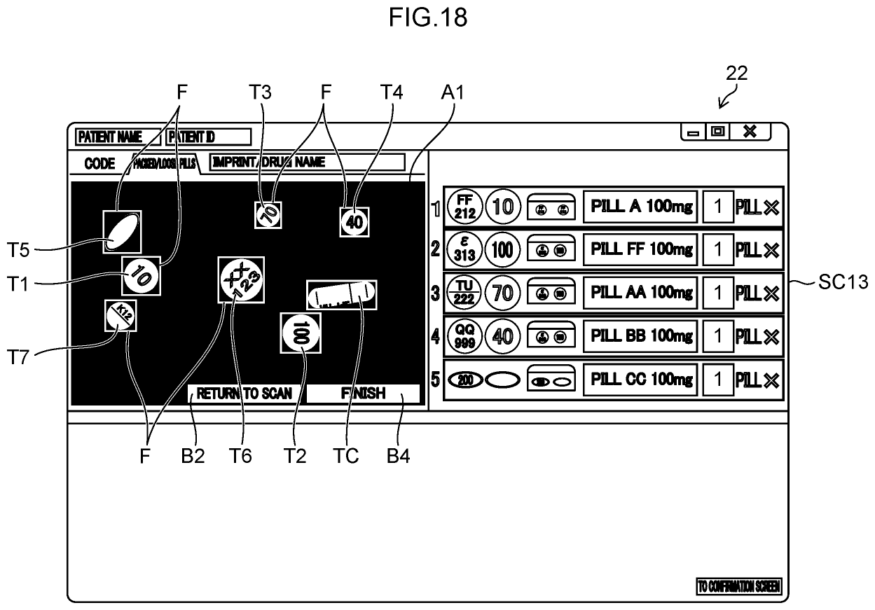

FIG. 18 shows a screen SC13 on the display 22 at this time point. As shown in FIG. 18, rectangular frames F that surround the areas of the seven tablets T1 to T7 and the capsule TC are displayed on the screen SC13. The imprints on the tablets T1 to T7 are displayed in an emphasized manner.

Next, the drug identifying apparatus 26 obtains, from the second image, a plurality of second clipped images by clipping areas where the respective drugs are displayed (step S10: example of second clipping step). The drug identifying apparatus 26 selects from the plurality of second clipped images, second clipped images for which it can be confirmed from the second clipped images themselves, that the second clipped images are images of drugs whose correct drugs have been determined in the first clipped images, and associates the selected second clipped images with the corresponding first clipped images (step S11: example of association step). Here, the drug identifying apparatus 26 confirms that the second clipped images correspond to the drugs for which correct drugs have been determined in the first clipped images, based on at least one of printed marks or imprints on the drugs, colors of the drugs, and shapes of the drugs on the second surface.

Furthermore, the drug identifying apparatus 26 displays the second clipped images, instead of displaying of the first clipped images, in a list form in a certain order in the first area A1 on the display 22, in such a manner that the user can respectively designate the displayed images (step S12: example of clipped image displaying step).

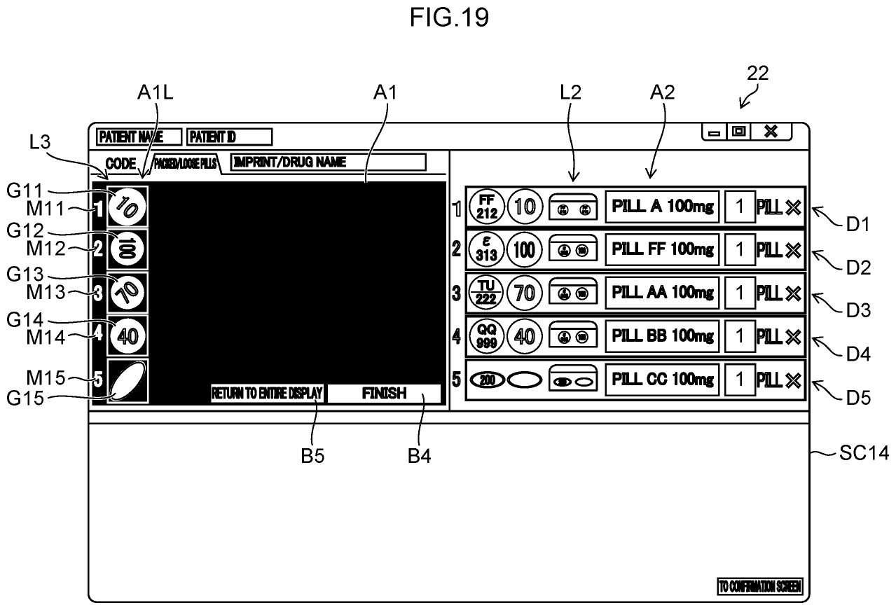

FIG. 19 shows a screen SC14 on the display 22 at a time point when the process in step S12 is finished. As shown in FIG. 19, in the left area A1L in the first area A1 on the screen SC14, the plurality of second clipped images are arranged side by side in the vertical direction of the display 22 and displayed in a list L3. Here, the second clipped images are associated with the respective information on the correct drugs in the list L2. Further, the second clipped images G11, G12, G13, G14 and G15, which respectively correspond to the tablets T1, T2, T3, T4 and T5, are displayed in the list L3.

On the screen SC14, the second clipped images in the list L3 and information on the correct drugs in the list L2, which respectively correspond to the second clipped images in the list L3, are arranged side by side in the lateral direction of the display 22. On the screen SC14, marks indicating the numbers according to the certain order are displayed adjacent to the respective second clipped images. Specifically, the second clipped images G11, G12, G13, G14 and G15 are assigned the respective marks M11, M12, M13, M14 and M15 which sequentially indicate numbers of 1, 2, 3, 4 and 5.

Furthermore, the drug identifying apparatus 26 identifies designated drugs which are designated by the user among the drugs of the second clipped images displayed in a list form on the display 22 (step S13). For example, the user designates a second clipped image among the displayed second clipped images in a list form, using the pointing device of the input apparatus 20. The drug identifying apparatus 26 identifies the designated drug, based on the second clipped image designated through the pointing device.

Subsequently, the drug identifying apparatus 26 obtains, from the drug information database 24, information on candidate drugs to determine a correct drug for the designated drug identified in step S13 (step S14). Here, the drug identifying apparatus 26 extracts three candidate drugs, and obtains information on the extracted three candidate drugs from the drug information database 24.

Next, the drug identifying apparatus 26 displays, in the first area A1 on the display 22, information that is on the candidate drugs, which are obtained in step S14, to determine the correct drug for the identified designated drug (step S15: example of candidate information displaying step).

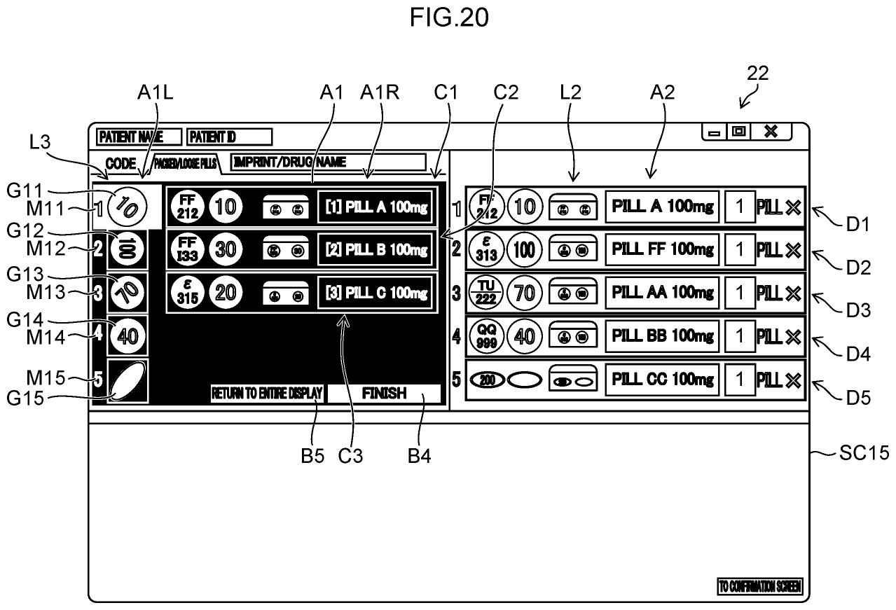

FIG. 20 shows a screen SC15 on the display 22 at a time point when the process in step S15 is finished. On the screen SC15, the second clipped image G11 of the tablet T1 is selected by the user, and the tablet T1 is identified as a designated drug. The periphery of the second clipped image G11 of the tablet T1, which is the designated drug, is displayed with a color different from the colors of the other parts in the first area A1, thus clearly indicating that the tablet T1 is the designated drug.

Furthermore, on the screen SC15, information on a plurality of candidate drugs for the tablet T1, which is the designated drug, is displayed in the right area MR in the first area A1 on the display 22 so as not to overlap the list L3. In the example of the screen SC15, information on the candidate drugs C1, C2 and C3 is included as the information on the candidate drugs.

On the screen SC15, the user can select information on one of the three candidate drugs C1, C2 and C3, using the pointing device of the input apparatus 20. Note that, as for the tablet T1, the correct drug has already been identified based on the captured image of the first surface. Accordingly, the user may select no correct drug if there is no need.

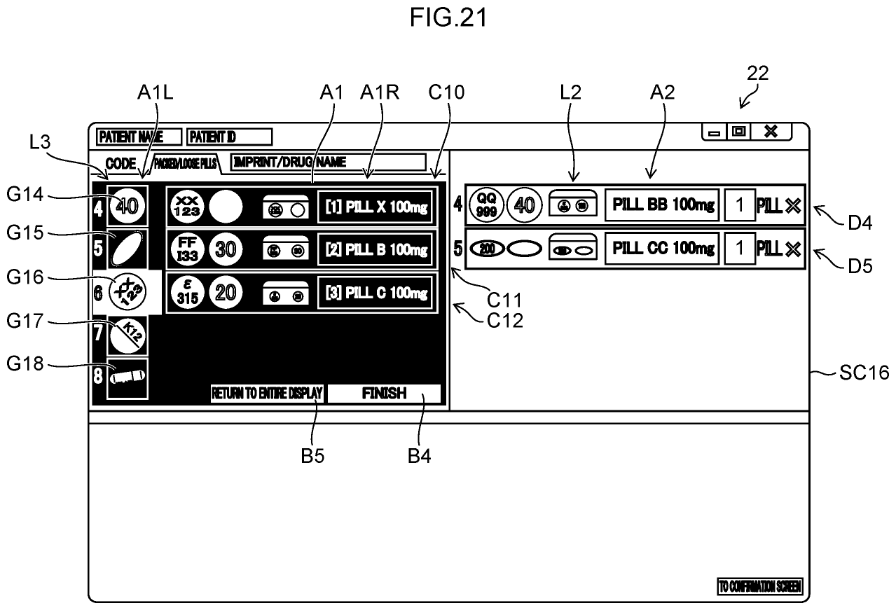

FIG. 21 shows a screen SC16 on the display 22 at a time point when the user scrolls the list L3 upward on the display 22 using the input apparatus 20 in a state where the screen SC15 is displayed. In the screen SC16, the list L3 is scrolled such that the second clipped images G11, G12 and G13 are hidden from the first area A1, and the second clipped images G14, G15, G16, G17 and G18 are displayed. Here, the list L2 is scrolled by the same amount in synchronization with the scrolling of the list L3. Accordingly, the second clipped image G15 and the information on the correct drug D5 corresponding to the second clipped image G15, are arranged side by side in the lateral direction of the display 22. Accordingly, the second clipped image and the information on the correct drug can be displayed in a contrasted manner.

On the screen SC16, the second clipped image G16 of the tablet T6 is selected by the user, and the tablet T6 is identified as a designated drug. The periphery of the second clipped image G16 of the tablet T6, which is the designated drug, is displayed with a color different from the colors of the other parts in the first area A1, thus clearly indicating that the tablet T6 is the designated drug.

Furthermore, on the screen SC16, information on a plurality of candidate drugs for the tablet T6, which is the designated drug, is displayed in the right area A1R in the first area A1 on the display 22 so as not to overlap the list L3. In the example of the screen SC16, information on the candidate drugs C10, C11 and C12 is included as the information on the candidate drugs. On the screen SC16, the user can select information on one of the three candidate drugs C10, C11 and C12, using the pointing device of the input apparatus 20.

The drug identifying apparatus 26 determines, as a correct drug corresponding to the designated drug, the candidate drug selected by the user from among the candidate drugs (step S16: determining step). Furthermore, in a case where correct drugs have been respectively determined for a plurality of second clipped images, the drug identifying apparatus 26 displays information on the correct drugs determined for the respective second clipped images, in a list form in the second area A2 on the display 22 (step S17: example of drug information displaying step). Here, the drug identifying apparatus 26 displays the second clipped images and the respectively corresponding information on the determined correct drugs, in association with each other. The drug identifying apparatus 26 displays information which is respectively associated with both the second clipped images and the information on the correct drugs (example of determining step).

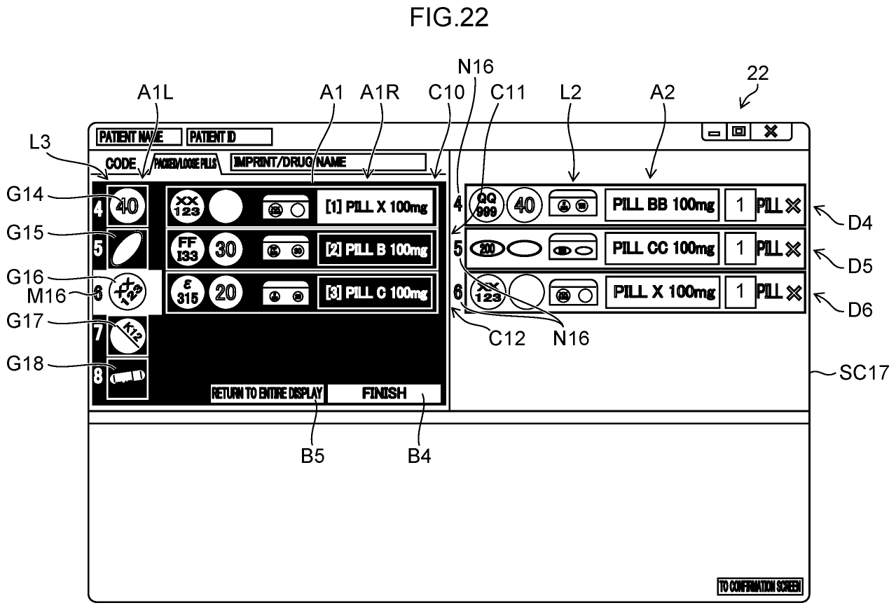

FIG. 22 shows a screen SC17 on the display 22 at a time point when the process in step S17 is finished. As shown in FIG. 22, on the screen SC17, a color of a field of a drug name of the candidate drug C10 selected by the user is changed.

On the screen SC17, the candidate drug C10 selected by the user is displayed as a correct drug D6 in the list L2 in the second area A2 on the display 22. On the screen SC17, the second clipped image G16 of the tablet T6, which is the designated drug, and the information on the correct drug D6, which is the correct drug corresponding to the tablet T6, are arranged side by side in the lateral direction of the display 22. Furthermore, on the screen SC17, a mark N16 indicating a number corresponding to a number indicated by a mark M16 which is assigned to the tablet T6, is displayed for the correct drug D6 in the second area A2. Accordingly, the second clipped image and the information on the correct drug can be displayed in a contrasted manner.

Subsequently, when the user operates the button B5 arranged at the bottom of the first area A1 using the input apparatus 20, the drug identifying apparatus 26 completes determination of the correct drugs for the second image, and finishes the processing of this flowchart.

Note that in a case where the drug package TP are turned upside down, a tablet T may be overturned within the drug package TP in some cases such that a face without any imprint on the tablet T may be imaged in both the first image and the second image. In this case, the drug package TP may be further overturned and similar procedures may be repeated. In this case, because the other tablets T have already been identified, the tablet T concerned can be easily identified.

In some cases, information which can be used to identify a drug T, is engraved only on one face of the drug T and no imprint is provided on the other face of the drug T. In a case where such a drug T is included in the drug package TP, identification of correct drugs is performed for identifiable drugs T when the drug package TP is imaged, once. After that, the imaging direction is changed (the drug package TP is turned upside down) and imaging is performed again, and drugs T are identified. In a case where the drugs T are identified after the drug package TP is turned upside down, if a positional relationship between the drugs T changes before and after the turning over of the drug package TP, it may be difficult to recognize the drugs T which have been identified in the first imaging or recognize the number of drugs T which are required to be identified in the second imaging, in some cases.

According to this embodiment, when the drugs T are identified from the second image in which the second surface of the drug package TP is imaged, the correct drugs identified from the first image in which the first surface is imaged, are displayed in the list L2. Therefore, the user may easily recognize the drugs T identified from the first image and the number of remaining drugs T which are required to be identified in the second image.

Note that among the processes in the drug identifying method, the processes in steps S1 to S8 constitute the drug list display method.

The technical scope of the present invention is not limited to the range described in the aforementioned embodiments. The configurations and the like of the embodiments may be appropriately combined among the embodiments in a range without departing from the spirit of the present invention.

### REFERENCE SIGNS LIST

10 . . . Drug identifying system
12 . . . Imaging apparatus
14 . . . Camera
16, 16A-16D . . . Light source
18 . . . Stage
18A . . . Mounting surface
20 . . . Input apparatus
22 . . . Display
24 . . . Drug information database
26 . . . Drug identifying apparatus
28 . . . Processor
30 . . . Memory
A1 . . . First area
A2 . . . Second area
A3 . . . Third area
B1-B5 . . . Button
C1-C12 . . . Candidate drug
D1-D6 . . . Correct drug
F . . . Frame
G1-G7 . . . First clipped image
G11-G19 . . . Second clipped image
M1-M7 M11-M19 . . . Mark
N1-N6 . . . Mark
SC1-SC15 . . . Screen
T . . . Drug
T1-T7 . . . Tablet
TC . . . Capsule
TP . . . Drug package
S1-S17 . . . Steps of drug identifying method

What is claimed is:

1. A drug list display method, comprising:
a first image obtaining step of obtaining a first image of drugs captured from a side of one surface of the drugs;
a first clipping step of clipping from the first image, areas where the respective drugs are displayed to obtain first clipped images;
a clipped image displaying step of displaying the first clipped images in a list form in a certain order in a first area on a display in such a manner that one or more of the first clipped images may be designated;
a determining step of determining correct drugs for first clipped images designated by a user; and
a drug information displaying step of displaying information on the correct drugs determined for the respective first clipped images in a second area on the display different from the first area, wherein the first clipped images and the corresponding information on the correct drugs are displayed in association with each other.

2. The drug list display method according to claim 1, wherein in the certain order, first clipped images which represent information contributing to determination of correct drugs are superior among the first clipped images, and first clipped images which represent no information contributing to determination of correct drugs are inferior.

3. The drug list display method according to claim 2, wherein among the first clipped images which represent the information contributing to determination of correct drugs, a first clipped image having a relatively higher degree of contributing to determination of the information becomes superior in the certain order.

4. The drug list display method according to claim 1, further comprising
a candidate information displaying step of displaying information on candidate drugs for determining the correct drugs for the one or more designated first clipped images,
wherein the determining step determines candidate drugs selected by the user from among the candidate drugs, as the correct drugs for the one or more designated first clipped images.

5. The drug list display method according to claim 4, wherein the candidate information displaying step displays the information on the candidate drugs in a third area between the first area and the second area on the display.

6. The drug list display method according to claim 4, wherein the information on the candidate drugs includes at least one of: names of the drugs; master images of the drugs;
and images of PTP (Press Through Package) sheets of the drugs.

7. The drug list display method according to claim 1, further comprising:
a second image obtaining step of obtaining a second image of the drugs captured from a side of another surface opposite to the one surface; and
a second clipping step of clipping areas of the respective drugs from the second image to obtain a second clipped images,
wherein the clipped image displaying step displays, instead of the first clipped images, the second clipped images in a list form in a certain order in such a manner that one or more of the displayed images may be designated.

8. The drug list display method according to claim 7, further comprising
an association step of selecting from among the second clipped images, one or more second clipped images for which it can be confirmed from the one or more second clipped images themselves, that drugs imaged in the one or more second clipped images are the correct drugs determined in the first clipped images, and associating the one or more second clipped images with the first clipped images,
wherein the clipped image displaying step displays the second clipped images corresponding to the first clipped images, and information on the correct drugs corresponding to the first clipped images, in association with each other.

9. The drug list display method according to claim 8, wherein the association step confirms drugs whose correct drugs are determined in the first clipped images, based on at least one of: printed marks or imprints on the another side of the drugs; colors of the drugs; and shapes of the drugs.

10. The drug list display method according to claim 7, wherein the determining step displays: the first clipped images or the second clipped images; and associated information which is associated with the first clipped images or the second clipped images and the information on the correct drugs.

11. The drug list display method according to claim 10, wherein the associated information includes a mark indicating a determined order.

12. The drug list display method according to claim 7, wherein the clipped image displaying step displays the printed marks and the imprints in the first clipped images and the second clipped images, in an emphasized manner.

13. The drug list display method according to claim 7, wherein in a case where the first clipped images or the second clipped images displayed in a list form in the first area are scrolled, the drug information displaying step scrolls the information on the correct drugs displayed in a list form in the second area in synchronization with the scrolling.

14. A non-transitory, computer-readable tangible recording medium which records thereon a program for causing, when read by a computer, the computer to execute the drug list display method according to claim 1.

15. A drug list to be displayed on a display, wherein:
first clipped images are displayed in a list form in a first area in a certain order, the first clipped images obtained by clipping areas where drugs are respectively displayed from a first image of the drugs captured from one side of the drugs;
information on correct drugs is displayed in a second area different from the first area, the correct drugs respectively determined for the first clipped images; and
the first clipped images and information on correct drugs respectively corresponding to the first clipped images, are displayed in association with each other.
